(19) United States
(12) Patent Application Publication
(10) Pub. No.: US 2025/0285763 A1
(43) Pub. Date: Sep. 11, 2025

(54) BIOLOGICAL AGE DETERMINATION USING A WEARABLE DEVICE

(71) Applicant: Apple Inc., Cupertino, CA (US)

(72) Inventors: Andrew MILLER, Brooklyn, NY (US); Christina HEINZE-DEML, Zollikon (CH); Guillermo R. SAPIRO, Durham, NC (US); Hamidreza ABBASPOURAZAD, San Diego, CA (US); Ian R. SHAPIRO, San Francisco, CA (US); Joseph FUTOMA, Brooklyn, NY (US); Matthew W. CROWLEY, Sunnyvale, CA (US); Saba EMRANI, Belmont, CA (US)

(21) Appl. No.: 19/054,773

(22) Filed: Feb. 14, 2025

Related U.S. Application Data
(60) Provisional application No. 63/563,288, filed on Mar. 8, 2024.
(60) Provisional application No. 63/652,876, filed on May 29, 2024.

(51) Int. Cl. G16H 50/30; G16H 40/63

(52) U.S. Cl. CPC G16H 50/30; G16H 40/63

(57) ABSTRACT

Aspects of the subject technology provide for training a machine learning model based on data from a healthy cohort of study participants. The machine learning model can be used to predict an age of a user based on physiological sensor data and determine a biological age of a user. An age gap can be determined between the user's chronological age and biological age and a notification or recommendation made to the user based on the age gap. An age gap rate of change can be made across multiple age gap determinations.

### CROSS REFERENCE TO RELATED APPLICATIONS

This application claims the benefit of U.S. Provisional Application No. 63/563,288, entitled “Biological Age Determination Using A Wearable Device,” filed Mar. 8, 2024, and U.S. Provisional Application No. 63/652,876, entitled “Biological Age Determination Using A Wearable Device,” filed May 29, 2024, the entirety of which are incorporated herein by reference.

### TECHNICAL FIELD

The present description relates generally to electronic devices, including, for example, wearable electronic devices with physiological sensors.

### BACKGROUND

Various physiological parameters of a user can be measured and analyzed to estimate other physiological measures indicative of the user's physiological state. Computer hardware has been utilized to make improvements across different industry applications including applications used to assess and monitor physiological activities.

### BRIEF DESCRIPTION OF THE DRAWINGS

Certain features of the subject technology are set forth in the appended claims. However, for purpose of explanation, several embodiments of the subject technology are set forth in the following figures.

FIG. 1 illustrates a diagram of various example electronic devices that may implement aspects of the subject technology in accordance with one or more implementations.

FIG. 2A illustrates a flow diagram for training a machine learning model in accordance with one or more implementations.

FIG. 2B illustrates a flow diagram for selection criteria of records for training a machine learning model in accordance with one or more implementations.

FIG. 2C illustrates a flow diagram for training a machine learning model in accordance with one or more implementations.

FIG. 3A illustrates an outcome of validation data for healthy population for a machine learning model in accordance with one or more implementations.

FIG. 3B illustrates an outcome of test data for general population for a machine learning model in accordance with one or more implementations.

FIGS. 4A, 4B, 5A, 5B, 6, 7A, and 7B illustrate various views of evaluations of a machine learning model on test data and validation data in accordance with one or more implementations.

FIG. 8 illustrates a flow diagram for predicting an age based on physiological signals in accordance with one or more implementations.

FIG. 9 illustrates a flow diagram for predicting an age based on physiological signals in accordance with one or more implementations.

FIG. 10 illustrates an example electronic system with which aspects of the subject technology may be implemented in accordance with one or more implementations.

### DETAILED DESCRIPTION

The detailed description set forth below is intended as a description of various configurations of the subject technology and is not intended to represent the only configurations in which the subject technology can be practiced. The appended drawings are incorporated herein and constitute a part of the detailed description. The detailed description includes specific details for the purpose of providing a thorough understanding of the subject technology. However, the subject technology is not limited to the specific details set forth herein and can be practiced using one or more other implementations. In one or more implementations, structures and components are shown in block diagram form in order to avoid obscuring the concepts of the subject technology.

Recent advancements in wearable device technology have led to the capability to record various physiological signals, which can be utilized for monitoring the overall wellness of users. Two of the most commonly collected physiological signals from wearable devices are photoplethysmography (PPG) and electrocardiogram (ECG). Wearable devices also collect motion signals, e.g., accelerometer, to augment and help filter such signals. Cardiac electrical activity is measured by ECG, containing information about cardiovascular health, while volumetric changes in arterial blood flow are measured by PPG, encompassing a wide range of biological information. Sensors for detecting these physiological signals may be employed on a wearable electronic device for the detection of certain health conditions, such as atrial fibrillation, and the monitoring of specific health metrics, such as, for example, blood oxygen. In some implementations, characteristics associated with arterial blood flow include heart rate, heart rhythm, heartbeat strength, or heartbeat timing.

Human biology is complex and interdependent such that physiological signals measuring one physiological trait may sometimes be used for deriving information about seemingly unrelated physiological traits. One problem traditionally facing researchers in this area is the lack of data. However, wearable devices having sensors to gather data from such signals are typically worn by users for extended periods of time. As a result, the amount of collected physiological data has grown.

Aspects of the subject technology utilize signals from a wearable device to predict a biological age of a user. A person's biological age can be understood to be the effective age of the person's physiology as impacted by health considerations which may positively or negatively impact the person's health. For example, if a person had no health considerations, then their biological age would approximately match their chronological age (e.g., their age measured from their date of birth). If a person has negative health issues, their biological age may appear to be older than their chronological age, whereas if a person is healthier than would be typical, their biological age may appear to be younger than their chronological age. For example, a person with health issues may be physiologically older than their chronological age, that is, produce physiological markers which can be measured by sensors which have characteristics of a person older than their chronological age.

The signals captured from the wearable device may be the physiological signals for PPG and ECG, as noted above, or in some instances may be other signals, such as from an accelerometer, not normally considered “physiological signals” but which may be used in the techniques described below. In one or more implementations, the recorded PPG signals are sampled at 64 Hz or 256 Hz, and may include four separate optical channels corresponding to different spatial combinations of transmitting and receiving diodes. The PPG segments may be pre-processed using dark subtraction (to reject signals introduced by ambient light), followed by bandpass filtering, down-sampling to 64 Hz (if needed) and temporal channel-wise z-scoring.

For the sake of simplicity, the physiological signals or the other signals that may be used in a similar manner, may together be referred to herein as physiological signals. The biological age of the user is predicted by comparing the physiological signals of the user to a trained machine learning model. In one or more implementations, data from a large group of study participants is prefiltered into a subset based on a cohort of study participants. The subset of data from the study participants can be further limited into a training set and a validation set. The training set is used to train the machine learning model by utilizing physiological signal data and the validation set is used to validate the model. Physiological signal data from the remaining study participants outside the subset may also be used for testing the machine learning model.

At least some of a human's physiological signal data will change as they age. Thus, after training the model, the model may be used on a user's physiological signal data to generate a prediction of the user's (biological) age, essentially based on a learned comparison of the user's physiological signal data with that of the physiological signal data of the trained model. By adjusting the prefiltering to establish the subset for training the machine learning model, one can likewise adjust the comparison. In some aspects of the subject technology, the prefiltering is performed to indicate that the training set more likely than not constitutes healthy individuals. If one presumes that healthy individuals age biologically at the about same rate as their chronological age, then the trained model can be used to compare a user's physiological signal data to the trained model to determine a prediction of age as compared to a group of healthy individuals. The predicted age can be considered the user's biological age. The biological age of the user can be compared to the chronological age of the user to determine if the user has an age gap between the chronological age (the actual age of the user in years) and the biological age (the predicted age of the user−the age of the user's biology or body based on their measured physiological signals). The age gap can be used as a general indicator of overall health of the user. The age gap can also be used to help guide the user into taking action to seek corrective behaviors or to make health decisions, for example, with the guidance of a doctor. Over time, the user can compare historical age gap data to determine a rate of change of the user's age gap.

Implementations of the subject technology improve the ability of a given electronic device to provide sensor-based, machine-learning generated feedback to a user (e.g., a user of the given electronic device). For example, the feedback may include age gap data for the user based upon data from the user collected at the electronic device. These benefits therefore are understood as improving the computing functionality of a given electronic device, such as an end user device which may generally have less computational and/or power resources available than, e.g., one or more cloud-based servers, and can be used to process the user's physiological signals in a privacy-preserving manner. Additionally, aspects of the subject technology utilize a machine learning model that is particularly trained by prefiltering data and physiological signal sensor data that is collected by a user's device to improve the ability to predict a person's age based on physiological signal data of the user. The subject technology further improves use of the user's electronic device by utilizing the electronic device to both determine age gap and provide age gap information to the user, as well as to monitor the user for a change in age gap over time. The subject technology further improves the use of physiological signals in a machine learning model by correlating biological age and chronological age as an indicator of health or disease in a manner that utilizes observations identified by a machine learning model which are imperceptible or not fully understood by humans.

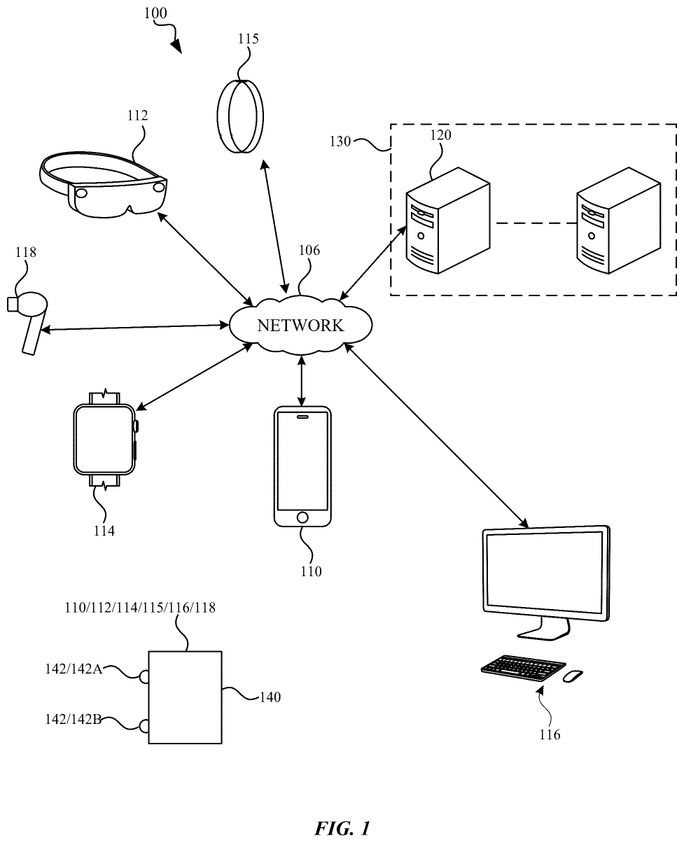

FIG. 1 illustrates an example network environment 100 in accordance with one or more implementations. Not all of the depicted components may be used in all implementations, however, and one or more implementations may include additional or different components than those shown in the figure. Variations in the arrangement and type of the components may be made without departing from the spirit or scope of the claims as set forth herein. Additional components, different components, or fewer components may be provided.

The network environment 100 includes an electronic device 110, an electronic device 112, an electronic device 114, an electronic device 115, an electronic device 116, an electronic device 118, and a server 120. The network 106 may communicatively (directly or indirectly) couple the electronic device 110 and/or the server 120. In one or more implementations, the network 106 may be an interconnected network of devices that may include, or may be communicatively coupled to, the Internet. For explanatory purposes, the network environment 100 is illustrated in FIG. 1 as including the electronic device 110, the electronic device 112, the electronic device 114, the electronic device 115, the electronic device 116, the electronic device 118, and the server 120; however, the network environment 100 may include any number of electronic devices and any number of servers or a data center including multiple servers. In some implementations one or more of the electronic devices 110-118 may not be connected to the network 106, but may be tethered to one of the other electronic devices 110-118 wirelessly or by a wired connection.

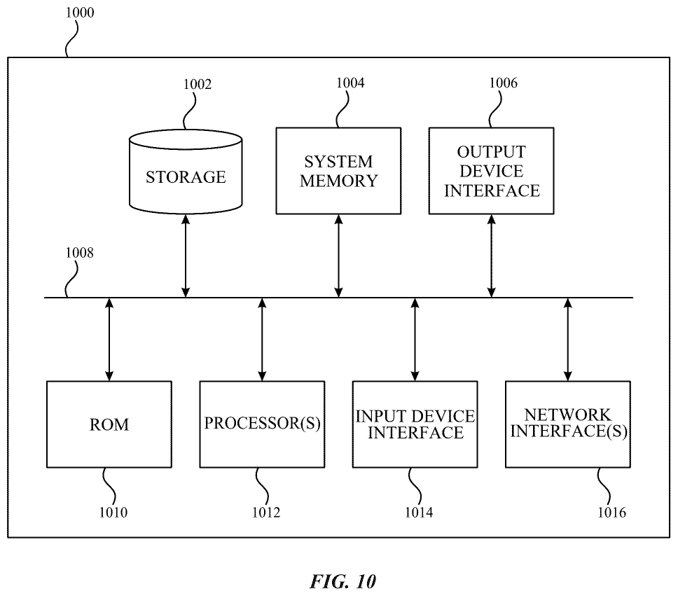

The electronic device 110 may be, for example, a desktop computer, a portable computing device such as a laptop computer, a smartphone, a peripheral device (e.g., a digital camera, headphones), a tablet device, a wearable device such as a watch, a band, and the like. In FIG. 1, by way of example, the electronic device 110 is depicted as a mobile electronic device (e.g., smartphone). The electronic device 110 may be, and/or may include all or part of, the electronic system discussed below with respect to FIG. 10.

The electronic device 112 may be, for example, desktop computer, a portable computing device such as a laptop computer, a smartphone, a peripheral device (e.g., a digital camera, headphones), a tablet device, or a wearable device such as a head mountable portable system, that includes a display system capable of presenting a visualization of an extended reality environment to a user. In FIG. 1, by way of example, the electronic device 112 is depicted as a head mountable portable system. The electronic device 112 may be, and/or may include all or part of, the electronic system discussed below with respect to FIG. 10.

The electronic device 114 may be, for example, desktop computer, a portable computing device such as a laptop computer, a smartphone, a peripheral device (e.g., a digital camera, headphones), a tablet device, a wearable device such as a watch, a band, and the like. In FIG. 1, by way of example, the electronic device 114 is depicted as a watch. The electronic device 114 may be, and/or may include all or part of, the electronic system discussed below with respect to FIG. 10.

The electronic device 115 may be, for example, desktop computer, a portable computing device such as a laptop computer, a smartphone, a peripheral device (e.g., a digital camera, headphones), a tablet device, a wearable device such as a watch, a band, and the like. In FIG. 1, by way of example, the electronic device 115 is depicted as a band. For example, the band may be worn on a wrist of a user. The electronic device 115 may be, and/or may include all or part of, the electronic system discussed below with respect to FIG. 10.

The electronic device 116 may be, for example, desktop computer, a portable computing device such as a laptop computer, a smartphone, a peripheral device (e.g., a digital camera, headphones), a tablet device, a wearable device such as a watch, a band, and the like. In FIG. 1, by way of example, the electronic device 116 is depicted as a desktop computer. The electronic device 116 may be, and/or may include all or part of, the electronic system discussed below with respect to FIG. 10.

The electronic device 118 may be, for example, desktop computer, a portable computing device such as a laptop computer, a smartphone, a peripheral device (e.g., a digital camera, headphones), a tablet device, a wearable device such as a watch, a band, and the like. In FIG. 1, by way of example, the electronic device 118 is depicted as an earbud. The electronic device 118 may be, and/or may include all or part of, the electronic system discussed below with respect to FIG. 10.

In one or more implementations, one or more of the electronic devices 110-118 may provide a system for training a machine learning model using training data, such as described herein. In one or more implementations, one or more of the electronic devices 110-118 may provide a system for utilizing a machine learning model after it has been trained to predict the biological age of a user based on the machine learning model and user data, such as described herein. Further, in one or more implementations, one or more of the electronic devices 110-118 may be a wearable device equipped with physiological sensors or other sensors for collecting data associated with a user of the device for use in comparing the data to a machine learning model for predicting the biological age of a user. Further, one or more of the electronic devices 110-118 may provide one or more machine learning frameworks for training machine learning models and/or developing applications using such machine learning models. In one or more implementations, training and inference operations that involve individually identifiable information of a user of one or more of the electronic devices 110-118 may be performed entirely on the electronic devices 110-118, to prevent exposure of individually identifiable data to devices and/or systems that are not authorized by the user.

The server 120 may form all or part of a network of computers or a group of servers 130, such as in a cloud computing or data center implementation. For example, the server 120 stores data and software, and includes specific hardware (e.g., processors, graphics processors and other specialized or custom processors) for rendering and generating content such as graphics, images, video, audio and multimedia files. In an implementation, the server 120 may function as a cloud storage server that stores any of the aforementioned content generated by the above-discussed devices and/or the server 120.

The server 120 may provide a system for training a machine learning model using training data, where the trained machine learning model is subsequently deployed to the server 120 and/or to one or more of the electronic devices 110-118. In an implementation, the server 120 may train a given machine learning model for deployment to a client electronic device (e.g., the electronic device 110, the electronic device 112, the electronic device 114, the electronic device 118). In one or more implementations, the server 120 may train portions of the machine learning model that are trained using (e.g., anonymized) training data from a population of users, and one or more of the electronic devices 110-118 may train portions of the machine learning model that are trained using individual training data from the user of the electronic devices 110-118. The machine learning model deployed on the server 120 and/or one or more of the electronic devices 110-118 can then perform one or more machine learning algorithms. In an implementation, the server 120 provides a cloud service that utilizes the trained machine learning model and/or continually learns over time. In an implementation, the server 120 and/or electronic devices 110-118 may utilize data collected at one of the electronic devices 110-118, anonymize the data, and update a machine learning model for deployment or use in other devices.

In the example of FIG. 1, each of the electronic devices 110-118 are depicted as a particular type of device, e.g., smartphone, head mounted portable system, smart watch, band, desktop or portable computer, and earbud. However, it is appreciated that each of the electronic devices 110-118 may be implemented as another type of device, such as a wearable device (e.g., a smart watch or other wearable device). The electronic devices 110-118 may be a device of a user (e.g., the electronic devices 110-118 may be associated with and/or logged into a user account for the user at a server).

Each of the electronic devices 110-118, may include a body or housing 140 containing elements such as input interfaces, output interfaces, processors, displays, processor(s), storage, system memory, read-only memory, network interfaces, and so forth, such as depicted in the electronic system discussed below with respect to FIG. 10. In particular, each of the electronic devices 110-118 may include input sensors 142, including one or more physiological sensors 142A collecting physiological signals and one or more other sensors 142B collecting other signals such as environmental or motion signals.

In one or more implementations, the physiological signals may include electromyography data recorded by at least one of the electronic devices 110-118, such as the electronic device 114, electroencephalography data recorded by at least one of the electronic devices 110-118, such as the electronic device 118, electrocardiography data recorded by at least one of the electronic devices 110-118, such as the electronic device 114, electrooculography data recorded by at least one of the electronic devices 110-118, such as the electronic device 118, and respiration data recorded by at least one of the electronic devices 110-118, such as the electronic device 118, among others. In one or more implementations, other signals recorded may be used to approximate physiological signals and can be used in place of or in addition to the aforementioned physiological signals and may include inertial motion sensor data, e.g., accelerometer data, recorded by at least one of the electronic devices 110-118. Additional signals may be used for other purposes, such as temperature data recorded by at least one of the electronic devices 110-118 and gyroscopic data recorded by at least one of the electronic devices 110-118.

To the extent that a single electronic device 114 is referenced herein, for the sake of simplicity, it is appreciated that any of the electronic devices of FIG. 1 may be utilized. Further, unless otherwise noted, it is appreciated that any of the physiological signals and/or other signals may be used when referring to physiological signals or physiological signals below. Any suitable techniques may be used in training the machine learning model.

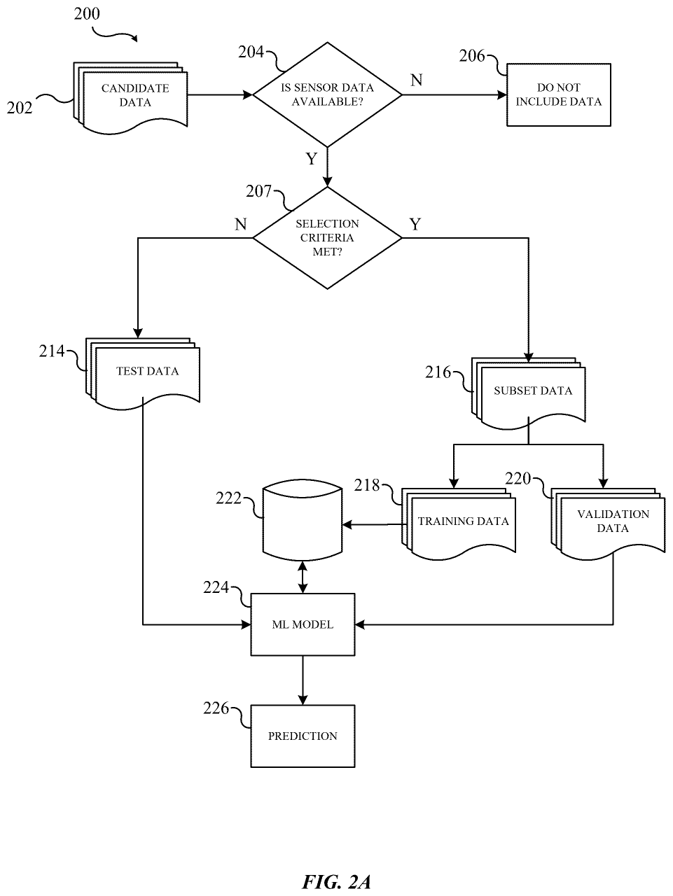

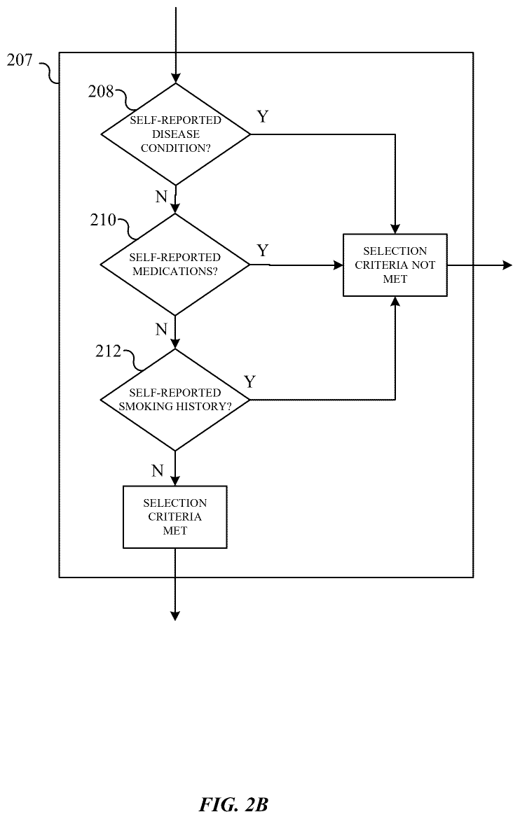

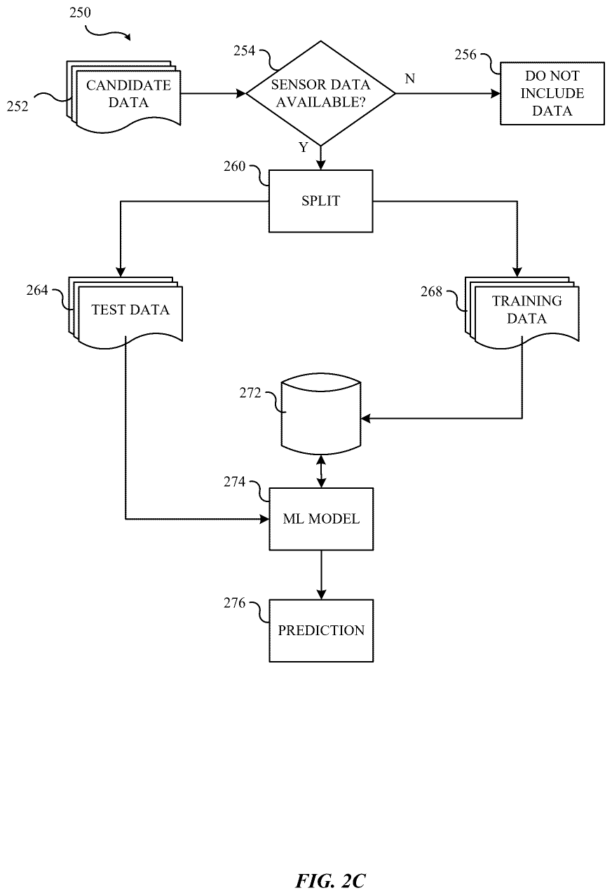

FIG. 2A illustrates a flow diagram of an example process 200 for training a machine learning model from a corpus of candidate data, in accordance with some implementations. The process 200 may be performed by one or more electronic devices, such as one of the electronic devices 110-118, described above with respect to FIG. 1. One or more blocks (or operations) of the process flow diagram of FIG. 2A may be performed by one or more other components and other suitable devices. Further for explanatory purposes, the blocks are described herein as occurring in serial, or linearly. However, multiple blocks of the process may occur in parallel. In addition, the blocks of the process need not be performed in the order shown and/or one or more blocks of the process need not be performed and/or can be replaced by other operations.

At block 202, candidate data is collected. In accordance with some implementations, the candidate data set may include data collected in a study from a group of participants. For example, one study may have a set of about 150,000 volunteer participants. The candidate data may include questionnaire data as well as input sensor data collected by, for example, a device of each candidate participant similar to those electronic devices 110-118 discussed above with respect to FIG. 1. The questionnaire data may be collected by means of administration of the questionnaire on an electronic device, such as one of the electronic devices 110-118, described above with respect to FIG. 1. The questionnaire may include questions to participants regarding self-reported behavioral and user data. The questionnaire data may be matched to the input sensor data and anonymized to protect privacy of the study participants. The questionnaire data may include a chronological age of the participant, whether the participant has been diagnosed or is self-diagnosed with a disease or adverse health condition, whether the participant takes regular prescription medications, whether the participant currently or has a usage history of smoking, drinking alcohol, or taking recreational drugs, and various information about the participant's activity and exercise level.

Other sources of health information may be used instead of or in addition to questionnaire data for candidate data. For example, some information may come from electronic health records that are submitted or accessed by permission from participants. It should be understood that accessing electronic health records may be performed in accordance with applicable laws and regulations and may be further protected by anonymization so as to remove identity information from any of the gathered data.

In one or more implementations, the candidate data includes data that is gathered over time based on health information stored and or generated at the one or more electronic devices 110-118, for example, for a multiple of varied users or participants. For example, one user of many different users may provide basic health-related information such as weight, height, and age at a profile associated with the one or more electronic devices 110-118. The one or more electronic devices 110-118 may collect indicators on health activity, such as indications of a healthy lifestyle, steps per day, miles per day walked, sedentary times, sustained heart rate elevation, respiratory rates, sleep data, heart rate variability, and so forth. These various indicators may be used to generally predict or determine that a user is likely to be healthy. The candidate data can correspond to data collected from multiples of such users.

At block 204, the candidate data can be examined to determine if enough or appropriate according to some quality selection criteria sensor data for physiological signals is available for each record for the purposes of creating, validating, or testing a machine learning model. For example, a minimum threshold of segments (collection periods of a pre-determined length) may be used to ensure that enough samples are available for a particular participant to achieve a meaningful result. The threshold, for example, may be between 5 and 50 segments, such as between about 10 and 20 segments, though other values are contemplated. The pre-determined length of each segment may be between about 1 sec and 300 secs, though other values are contemplated. In implementations that utilize a questionnaire, the segments for consideration may be selected from all available segments as those being within a set time period from which the questionnaire data was collected. For example, it may be desirable to only include segment data within 7, 15, 30, 45, 60, 75, or 90 days (though other values are contemplated) of the collection of the questionnaire data so that the segments are more likely to be collected when the questionnaire data is still valid and/or is more accurate. Similarly, it may be desirable to include segment data that overlaps the time that the questionnaire was completed such that at least some of the segment data comes before a completion date of the questionnaire. For example, as time goes on, participants might change relevant habits, be diagnosed with medical conditions, or have some other situations arise which would skew the segment data. The longer the time period from the time of the questionnaire that the segment data was collected, the more suggestive it is that the corresponding questionnaire data may be invalid or stale. The segments for consideration may also be selected or filtered based on a quality criteria, for example, when the subject was not moving as detected by inertia sensors.

In embodiments not relying on a questionnaire, physiological signals can be collected, along with the data collected from electronic health records or along with the activity data of participants.

At block 206, if enough sensor data segments are not available for a particular candidate record over the threshold and within the time period from the questionnaire, then these records from the candidate data are not included.

At block 207, the candidate data is analyzed if it meets a selection criteria. The selection criteria may be used to provide training data filtering for the purpose of supervised learning. In some implementations, selection criteria may be used to include generally only candidate data having particular characteristics. As discussed in further detail below, as an example such selection criteria may be used to determine if each candidate record corresponds to a healthy individual. The selection criteria used to approximate whether a candidate record corresponds to a healthy individual can be chosen as appropriate. As noted above, in some instances, height, weight, age, and some activity level information may be included with each record, and the same may be used to approximate a healthy person. For implementations using questionnaire data, questions may be included to participants to help determine healthiness. One such selection criteria is provided in FIG. 2B.

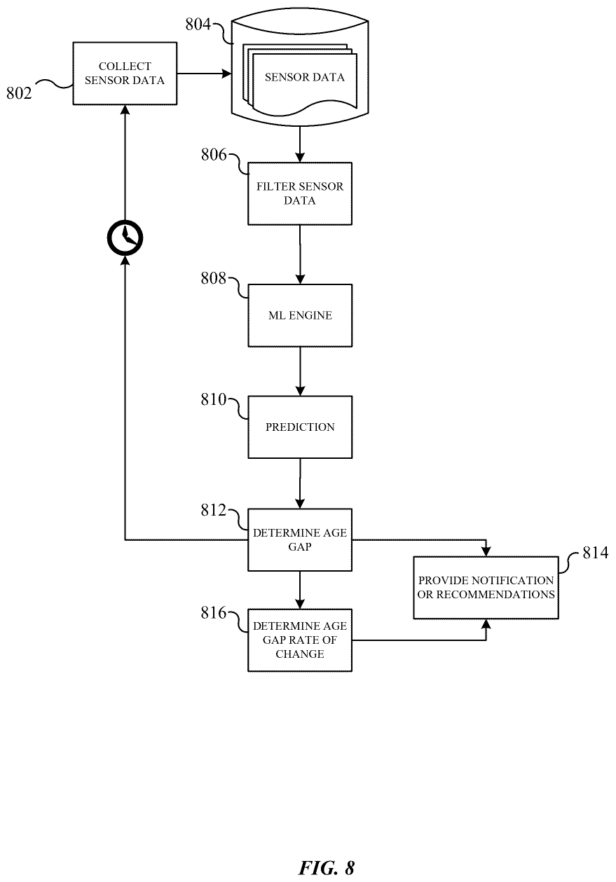

The selection criteria may be chosen so as to personalize the resulting training data for the model. The personalization may be based on a particular user whose physiological signal data will be provided to the model once trained, for example, as discussed below with respect to FIG. 8. For example, training data can include only those people of similar age as the particular user and having a similar height, but with at least one characteristic that is different than the particular user. When the candidate data is analyzed and determined whether the selection criteria is met, the resulting training data will be used to train a model that is customized to be more characteristic of the particular user.

Turning briefly to FIG. 2B, a selection criteria flow is provided as an example implementation of the block 207 of FIG. 2A, in accordance with some implementations. It should be understood that the selection criteria flow illustrated in FIG. 2B is merely an example and other criteria may be used instead of or in addition to the criteria discussed herein. It should also be understood that the flow of these elements may be provided for in a different order, and in some instances may be performed in parallel.

Blocks 208, 210, and 212 are discussed below in terms of self-reported data received from a questionnaire. It should be appreciated that each of data below may be determined by other means, for example, such as by electronic health records provided or accessed with permission. For example, disease conditions, medications taken, and/or smoking history for a user with a set of physiological signals may be obtainable for consideration by electronic health records rather than or in addition to questionnaire data.

At block 208, the questionnaire data can be analyzed to determine if the participant self-reported a disease condition. The disease conditions with which to filter may be selected from a list of recognized chronic or acute diseases, and may include, for example, one or more of heart disease, heart failure, diabetes, pacemaker usage, liver disease, arterial disease, stroke or transient ischemic attack, neuropathy, heart attack, high blood pressure, atrial fibrillation, osteoporosis, hip or knee problems, kidney disease, sleep apnea, arthritis, chronic bronchitis, neck disorder, depression, high cholesterol or genetic dispositions, heart rhythm, anxiety, urinary issues, lower back pain, cancer, thyroid function, vision function, hearing function diseases, asthma, or allergies. Other conditions may be selected. Models may be trained or retrained based on a determination that one or more of the disease conditions do not tend to affect or are discovered not to affect age prediction. For example, if allergies do not negatively impact biological age versus chronological age, then an allergy condition may not preclude a participant who has allergies from being considered for inclusion in the training data. If the participant has a qualified self-reported or otherwise obtained disease condition, then the participant's information can be included in the test data at block 214. If the participant does not have any qualified self-reported or otherwise obtained disease conditions, then the flow continues to block 210.

At block 210, the questionnaire data can be analyzed to determine if the participant self-reported the use of medications. In some implementations, any medications in use can be cross-referenced with a list of allowable or disallowable medications to determine if the participant should continue to be considered for inclusion in the training data. In some implementations, any reported medication use may cause the participant to be excluded from the training data. If a list of medications is used, over-the-counter vitamins and chemical pharmaceuticals may be allowable along with some prescribed medications such as hormonal birth control. If the participant has a qualified self-reported medications, then the participant's information can be included in the test data at block 214. If the participant does not have any qualified self-reported medications, then the flow continues to block 212.

At block 212, the questionnaire data can be analyzed to determine if the participant self-reported past or current smoking, alcohol consumption trends, and/or the use of recreational drugs. In some implementations, each of these considerations may have threshold based on usage frequency, usage history, and usage quantities, which may be used to qualify or disqualify the participant from being used in the training data. For smoking, for example, any use may be disqualifying or any use within the last x number of years, such as 10, 15, or 20 years, may be disqualifying. For alcohol consumption, an average number of alcoholic drinks per month in excess of another threshold may be disqualifying. Similarly, regular or habitual recreational drug use may be disqualifying. Each of these metrics may be individually determined and may be altered and the machine learning model retrained as needed. If the participant has a qualified self-reported smoking, alcohol, or recreational drug use, then the participant's information can be included in the test data at block 214. If the participant does not have any qualified self-reported smoking, alcohol, or recreational drug use, then the flow continues to block 216.

It should be appreciated that fewer or additional criteria may be used in addition to or instead of the criteria discussed above with respect to blocks 208, 210, and 212. It is understood that the filtering may filter out “healthy” candidates. The goal is to obtain a training data set that only includes “healthy” candidates, however, it is also understood that questionnaire data is inherently unreliable because it relies on self-reporting and there is a risk that participants do not self-report accurately whether intentional or not. Utilizing the training data on a machine learning model, however, will tend to focus good data and moderate bad data by virtue of how machine learning models work.

Returning to FIG. 2A, at block 216, the subset of data derived from the candidate data at block 202 may be randomly split into training data at block 218 and validation data at block 220. The split may be any suitable distribution, such as 90% training data and 10% validation data, 80% training data and 20% validation data, 70% training data and 30% validation data, 60% training data and 40% validation data, or 50% training data and 50% validation data, the reverse, or the like.

At block 222, the training data is included in a database or other storage mechanism for use by the machine learning model for training. The training data storage can be updated to add additional training data or remove training data from time to time. In such instances, the machine learning model can be altered or retrained to account for the updating of the training data. In some implementations, the machine learning model can be further trained with a particular user's physiological signal data. For example, a prediction from the machine learning model for the same user at a later time includes a consideration for the user's previous physiological signal data.

At block 224, a machine learning model is created based on the training data. Any suitable machine learning model training process may be utilized for training the machine learning model. By selecting a cohort of the corpus of available candidate data as being limited to those participants perceived to be or having a high likelihood as being healthy, the training of the model correlates the physiological signal data in the segment data with the participants characteristics provided for on the questionnaire, such as chronological age, biological sex, height, weight, and so forth. As such, the machine learning model may be unbounded to a ground truth, but because the training data is limited according to a specified characterization, predictions made against the machine learning model for biological age can provide a predicted biological age based on the physiological signals of a “healthy” individual as baseline. In implementations where the training data is selected based on other criteria, such as provided for in a personalized or customized training data set, the predictions made against the machine learning model for biological age can provide a biological age based on the physiological signals of the personalized or customized characteristics.

Returning for a moment to block 220, the subset data which was not used for training can be used for validation of the machine learning model. Often, machine learning models lack real world training and/or validation data and rely on manufactured training data and/or test data. As such, the machine learning model of the subject technology has advantages over other machine learning models as being more indicative of real world rather than idealized effects. This is also pertinent in the present context because humans are unable to perceive or understand how physiological signals change in relation to chronological age or biological age, and how exactly physiological signals, biological age and chronological age are related to health or disease.

Validation can involve utilizing the candidate data to use the machine learning model to predict the age associated with each record of the candidate data based on the physiological signals for that record. Since the validation data is also from “healthy” individuals (or individuals meeting the selection criteria), the predicted ages for the records of the validation data should be close to the actual age as provided for in the respective record of the validation data. As noted above, where the selection criteria is to determine healthy individuals, it is presumed that for a healthy individual included in the training data, the biological age of the healthy individual should be approximately the same age as the chronological age of the healthy individual.

Returning again to block 214, with regard to the candidate data that was rejected as not meeting the selection criteria block 207, e.g., as a result of the health determination reflected by blocks 208, 210, and 212, such candidate data can be used as test data against the machine learning model. Since this test data includes candidate data for individuals who were did not meet the selection criteria, e.g., not deemed as likely to be healthy, the test data can be used to see how chronological age and biological age diverge for users who may have health conditions. Undoubtedly, the test data also includes healthy individuals who just did not happen to be included in the training data. Thus, this test data does not necessarily indicate that each tested participant data is expected to have divergent biological age and chronological age indicative of a health condition.

At block 226, a prediction can be made based on the validation data and/or the test data by providing the data to the trained machine learning model and receiving a prediction from the machine learning model. For example, on new PPG segments, the trained machine learning model can output predictions that include PPGAge predictions (or predictive age), the PPGAge gap (or age gap), as well as longitudinal PPGAge time series. Candidate data records represented in the test data and validation data, however, include the information, including chronological age, provided by the questionnaire data, electronic health records, and/or other data sources.

In one or more implementations, for the validation data, the expectation is that the prediction for a particular validation case should be close to the corresponding chronological age. In one or more implementations, the validation data can be used to analyze the accuracy of the PPGAge predictions, its cross-sectional associations with disease and behavior, and its longitudinal sensitivity to physiological changes, such as pregnancy and cardiac events. Further, other information from the validation data can be used to adjust the machine learning model or find cross-correlated effects according to the candidate data. Similarly, for the test data, because the test data for the candidate data also contains the information, including chronological age, provided by the questionnaire data, electronic health records, and/or other data sources, divergent biological age and chronological age can be cross correlated with the self-reported health conditions or otherwise known medical conditions to determine which health conditions tend to impact biological age. Health conditions which do not impact biological age can be removed from concern, for example, at block 208 and a machine model retrained based on a different selection of participants from the candidate data.

FIG. 2C illustrates a flow diagram for training a machine learning model using self-supervised learning in accordance with one or more implementations. The process 250 may be performed by one or more electronic devices, such as one of the electronic devices 110-118, described above with respect to FIG. 1. One or more blocks (or operations) of the process flow diagram of FIG. 2C may be performed by one or more other components and other suitable devices. Further for explanatory purposes, the blocks are described herein as occurring in serial, or linearly. However, multiple blocks of the process may occur in parallel. In addition, the blocks of the process need not be performed in the order shown and/or one or more blocks of the process need not be performed and/or can be replaced by other operations.

At block 252, candidate data is collected. Candidate data can be collected utilizing any of the processes and materials such as those described above with respect to block 202, which are not repeated. In some implementations, the candidate data may entirely or mostly correspond to the user, for example, historical physiological signal data of the user.

At block 254, the candidate data can be examined to determine if enough or appropriate according to some quality selection criteria sensor data for physiological signals is available for each record for the purposes of creating, validating, or testing a machine learning model. The examination of the candidate data may be performed utilizing any of the processes and materials such as those described above with respect to block 204, which are not repeated.

At block 256, if enough sensor data segments are not available for a particular candidate record over the threshold and within the time period from the questionnaire, then these records from the candidate data are not included.

At block 260, the remaining candidate data may be randomly split into training data at block 268 and test data at block 264. The split may be any suitable distribution, such as 90% training data and 10% test data, 80% training data and 20% test data, 70% training data and 30% test data, 60% training data and 40% test data, or 50% training data and 50% test data, the reverse, or the like.

At block 272, the training data is included in a database or other storage mechanism for use by the machine learning model for training. The training data storage can be updated to add additional training data or remove training data from time to time. In such instances, the machine learning model can be altered or retrained to account for the updating of the training data.

At block 274, a machine learning model is created based on the training data. Any suitable machine learning model training process may be utilized for training the machine learning model. In some implementations, a self-supervised learning process is used to train the machine learning model. While self-supervised learning may result in subject-focused encodings, when the training data corresponds to historical user data, then the model can utilize the user's own data to predict biological age which may vary from the user's chronological age because of changes in health, medical procedures, and so forth. Further, the physiological characteristics of the physiological signals are learned through the self-supervised learning process. The model can provide a user-specific baseline from which to provide comparisons. Wearing a device such as described herein can provide many segments of physiological signal data from which to train the machine learning model. Some implementations may use self-supervised learning to train the machine learning model including physiological signal data from other users as well. In one or more implementations, the machine learning model may be a deep neural network such as an encoder that is trained from about 10 million unlabeled PPG segments via self-supervised learning (SSL), with the objective of distinguishing participants from each other. The machine learning model as a trained encoder can transform raw input data into a dense, informative representation or embedding. In one or more implementations, the machine learning model may be a neural network that takes 4-channel green PPG signal into a 256-dimensional feature vector called representation. For example, the machine learning model can summarize the information in a PPG segment with a 256-dimensional feature vector into a PPG representation (or PPG embedding). In one or more implementations, the PPG representation encodes significant amount demographic and health-related information of a population of users. In one or more implementations, the machine learning model may further include a neural network that undergoes representation learning by learning a linear prediction head (e.g., linear probing) from the pre-trained encoder to age as a target variable. For example, the machine learning model may be produced into a model of healthy aging by selecting data indicating self-reported healthy participants and fitting this selected data into a least squares linear regression model to predict chronological age from the average of PPG representations over a span of 30 days (with a minimum of 30 PPG segments, for example).

Returning to block 214, the test data from block 264 can be used to test the machine learning model. This test data was randomly set aside from the candidate data and so the prediction resulting from the test data should be approximately the same as actually reflected in the candidate data.

At block 276, a prediction can be made based on the test data by providing the test data to the trained machine learning model and receiving a prediction from the machine learning model.

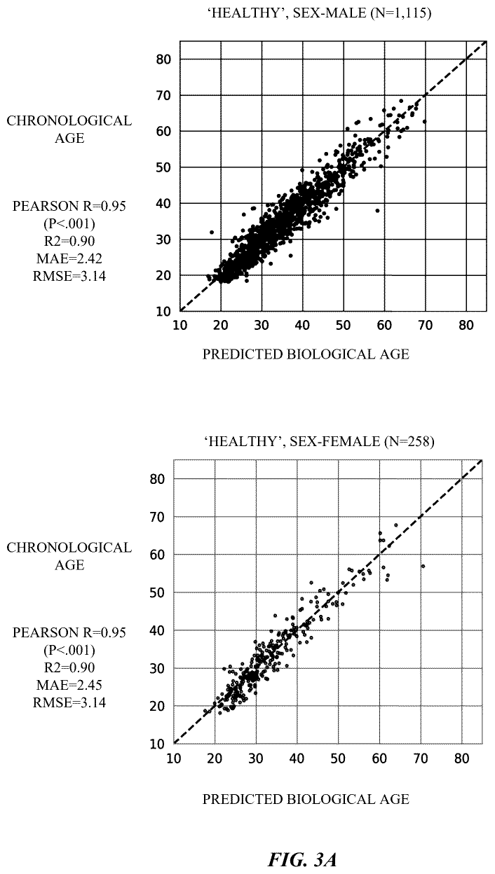

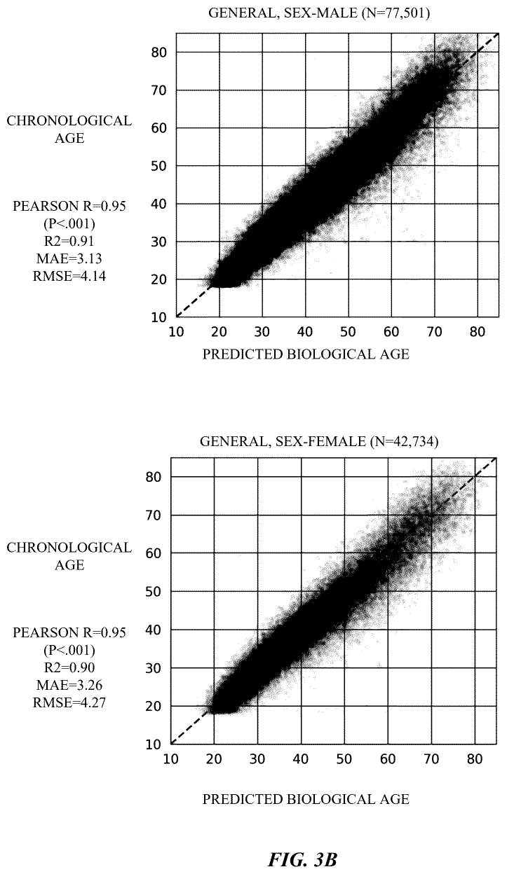

FIG. 3A illustrates validation data as described above. The plot on the left is for biological males and the plot on the right is for biological females. The diagonal on each plot is the line where the predicted age equals the biological age. Thus, ideally each of the predictions among the validation data (e.g., for “healthy” participants) should be right on the diagonal. One limitation in the data used in this example is that only the year of birth is known for participants, as a measure of privacy, so it is actually expected to see the kind of variability observed. However, to the extent that there is variability from the diagonal, the variability should be about evenly distributed above the diagonal, i.e., where the predicted biological age is less than the chronological age, and below the diagonal, i.e., where the predicted biological age is greater than the chronological age. And that holds true for the validation data, as observed in FIG. 3A.

FIG. 3B illustrates test data as described above. The plot on the left is for biological males and the plot on the right is for biological females. The diagonal on each plot is the line where the predicted age equals the biological age. Thus, ideally each of the predictions among the test data (e.g., for participants not included in the “healthy” participants group) should be on or below the diagonal. However, due to the limitations in the data used in this example, it is expected to observe predicted ages above the diagonal as well. As can be observed in the graphs of FIG. 3B, however, the bulk of the test data is below the diagonal, as is expected. It is also noted that as people age, the number of participants who are not on medications or do not have self-reported health issues is diminished such that the reliability of that data is questionable for two reasons. First, there is just less data since many if not most people in general will have some health complications or take medicine to avoid health complications. Second, to the extent that an aged participant self-reports that they do not have health conditions or take medications, the participants may be mistaken. As a result, above the age of about 55 or 60 the test data skews above the line, indicating that the predicted biological ages are more likely less than the chronological ages. To address this aspect of the subject technology, multiple models may be used which use different training data with different based on an assumption that a “healthy” older person is expected to take certain medications or have dealt with certain disease diagnoses.

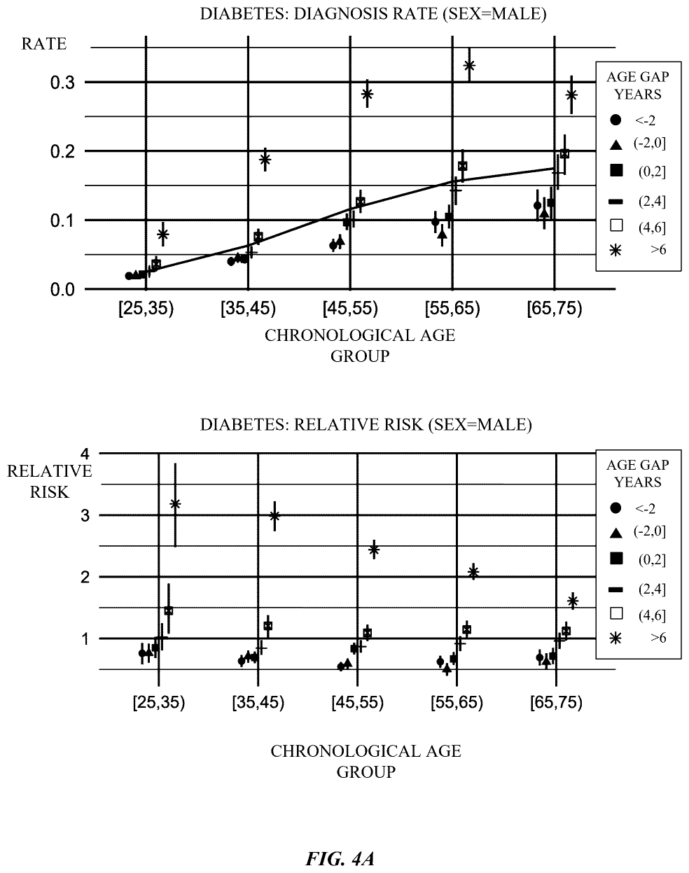

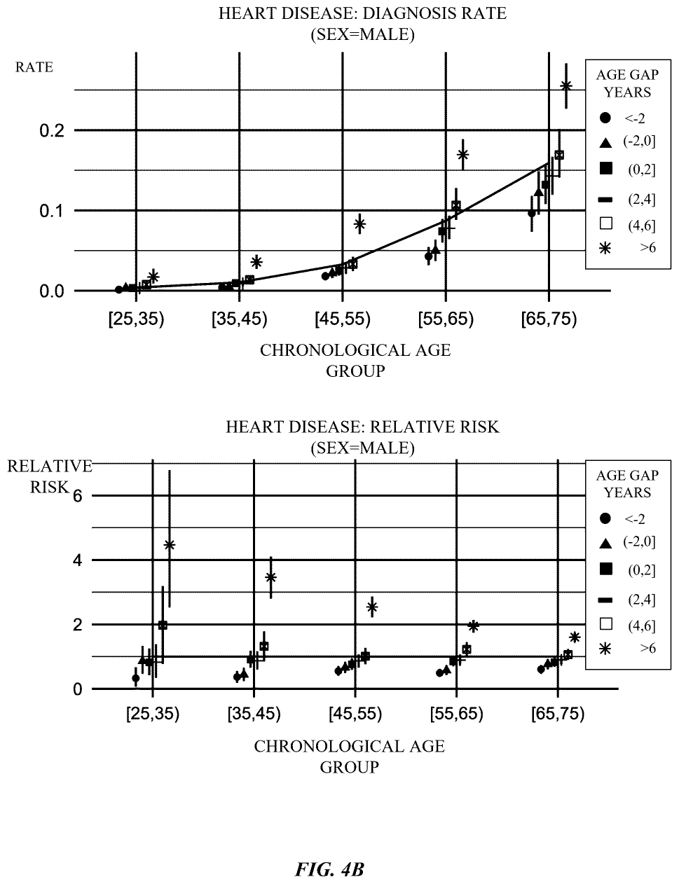

FIG. 4A illustrates an evaluation of how diabetes as a disease correlates to age gap in biological males. The plot on the top groups test participants by chronological age and provides a diagnosis rate for diabetes. The average diagnosis rate is provided by the dashed line. The average rate of diagnosis by age gap range is provided by the plot points, where 0 looks oldest and 4 looks youngest. This data is then cross correlated on the plot on the bottom which shows that the relative risk of having diabetes as it pertains to biological age is directly related to the diagnosis rate. This indicates that the diagnosis of diabetes is highly correlative to age gap.

FIG. 4B illustrates an evaluation of how heart disease correlates to age gap in biological males. As can be seen on the top plot, the level of diagnosis of heart disease goes up among all populations with age. As a result, as can be seen in the bottom hand plot the diagnosis of heart disease is highly correlative to age gap among younger individuals, but may become less correlative in older individuals.

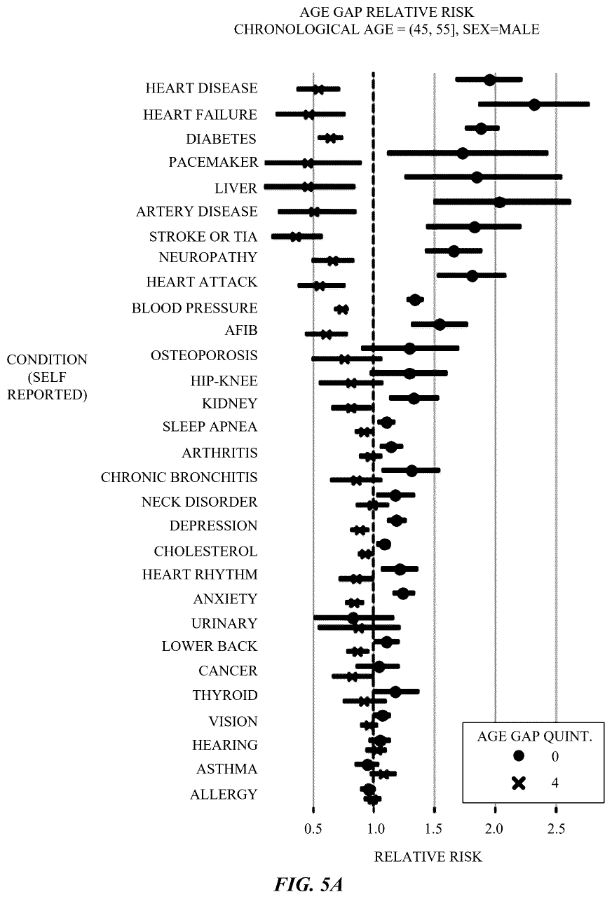

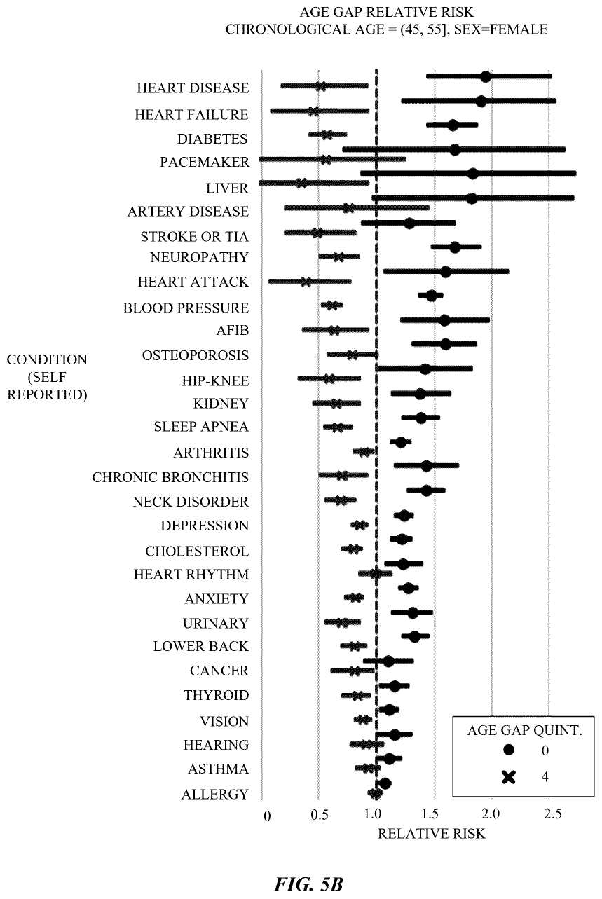

FIGS. 5A and 5B illustrate an evaluation of the relative risk of various diseases to affect age gap. FIG. 5A provides this data for risk in biological males and FIG. 5B provides this data for risk in biological females. The diseases are roughly sorted by those having large apparent effects on age gap to those that have small apparent effects on age gap.

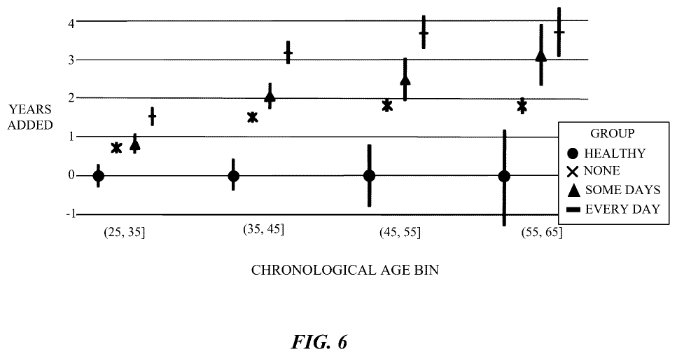

FIG. 6 illustrates an evaluation of the predicted age gap separated by age cohorts for participants in the test data who smoke some days, smoke every day, or not at all. As can be seen in the illustration of FIG. 6, for example, in the 35-45-year-old cohort, smoke is associated with a 2-year age gap and daily smoking is associated with an age gap of more than three years. Similar observations can be made for smokers in other age cohorts.

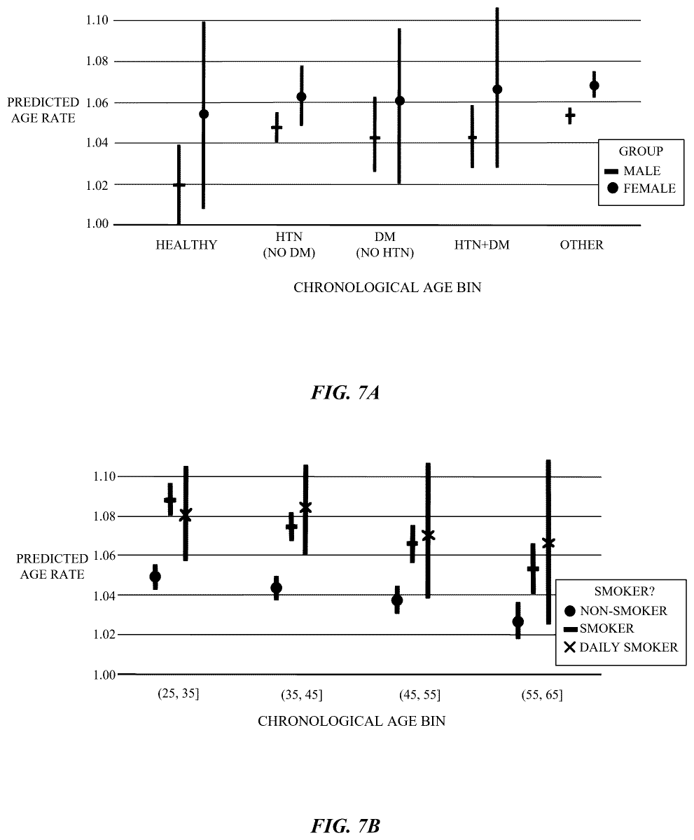

FIG. 7A illustrates an evaluation of the predictive power of using biological sensor data to predict age gap among test and validation participants for biological males and biological females. As can be seen in data, age gap predictions correlate to the self-reporting of disease and/or medication. That is, predicted age gap increases correlatively to when a participant reports disease or medication.

FIG. 7B illustrates an evaluation of the predictive power of using biological sensor data to predict age gap among test and validation participants for non-smokers, smokers, and daily-smokers. As can be seen in data, age gap predictions correlate to the self-reporting of smoking. That is, predicted age gap increases correlatively to when a participant reports that they are a smoker.

FIG. 8 illustrates a flow diagram of an example process 800 for utilizing a machine learning model to predict biological age of a user based on physiological signals, in accordance with some implementations. One or more blocks (or operations) of the process flow diagram of FIG. 8 may be performed by one or more other components and other suitable devices. Further for explanatory purposes, the blocks are described herein as occurring in serial, or linearly. However, multiple blocks of the process may occur in parallel. In addition, the blocks of the process need not be performed in the order shown and/or one or more blocks of the process need not be performed and/or can be replaced by other operations.

At block 802, sensor data can be collected at a wearable device. The wearable device, for example, may correspond to one of the electronic devices 110-118 as described above with respect to FIG. 1. The sensor data may correspond to the physiological signals previously mentioned, that is, for measuring PPG data or ECG data. Other signals from other sensor data may also be used which may approximate physiological sensor data. For example, an accelerometer located near a vein or artery may register movement of blood through the vein or artery, e.g., a heartbeat, in a manner that in a sense approximates a PPG sensor. PPG can measure blood flow and an accelerometer located near a vein or artery may measure the slight movement in accordance with a user's heartbeat and the flow of blood. Therefore, although a PPG sensor would provide a better source of data than an accelerometer for blood flow, an accelerometer may be used in a similar manner as the PPG sensor. The sensor data may be collected on the wearable device and stored for processing by an age prediction process running on the wearable device or on another electronic device, such as the electronic devices 110-118. In such implementations, the sensor data can be seen as being collected from the wearable device or from a place of storage of the data.

At block 804, the sensor data may be stored in a database or other storage structure, for example, in flash memory, SDRAM, ROM, or other computer-readable memory. The sensor data may be collected and/or stored in segments of sensor data having a length between about 1 second and 500 seconds, though other values are contemplated and may be used.

At block 806, the sensor data may optionally be processed to filter the sensor data to remove data that may be deemed to be unreliable. For example, the sensor data is ideally collected and compared when the user is at rest and has been at rest for some time, so that a baseline result can be achieved for each of the sensor data segments. The filter may be based on an accelerometer or other movement such as detected by an inertial measurement unit. The filter may also include ambient temperature readings and/or body temperature readings to filter out segments that might have been taken when the user was in an abnormally cold or hot environment or was running a fever.

At block 808, the sensor data or the filtered sensor data is provided to the ML Engine, such as the trained machine learning model discussed above. The ML Engine utilizes the data to predict or estimate what the age is of the person whose data was provided. The ML Engine may have separable models based on some user attributes, such as biological sex, or as discussed previously, based on chronological age being above a certain threshold. The ML Engine may be stored and utilized on the wearable electronic device where the sensor data is collected or may be stored and utilized on another electronic device. If the ML Engine is used at another device, it is understood that best practices may be utilized to protect the privacy of personal data of the user of the wearable electronic device.

At block 810, the ML Engine provides a predicted age. In some implementations, the predicted age is taken to correspond to an equivalent biological age. In other implementations, the determined biological age may be based on the predicted age, but may be adjusted downward or upward depending on considerations which may be discovered at a later time. For example, if a separate model is not used for older people, an algorithm can be developed to add a scalar value of the chronological age to the predicted age to arrive at the biological age. Or in another example, if it is discovered that people from certain countries have predicted ages that are consistently under or over what the prediction should be, the biological age may be an offset of the predicted age for people in those countries.

At block 812, the age gap is determined by subtracting the chronological age (the user's actual age) from the biological age (based on the predicted age). If the age gap is positive, then that indicates that user appears to be biologically older than they actually are. If the age gap is negative, then that indicates that the user appears to be biologically younger than they actually are.

At block 814, a notification or recommendation can be provided to the user based on the age gap. The notification can be at the wearable device in some implementations or may be by another communications method in accordance with other implementations. In some implementations, the age gap can be correlated with other known data about the user. For example, if the age gap is high and the user is overweight or not active, has had a health condition, or has been taking medication, then the age gap can be correlated to one or more of these conditions. The correlation may be, for example, for the purpose of providing recommendations to the user based on the age gap and the correlated information. For example, a recommended intervention may be provided for healthy meals, meal timing, exercises, exercise encouragement, doctor intervention, and so forth. In some implementations, if the biological age is above a recommended age for a preventative medical test, a recommendation may be provided to the user to consult with a doctor about obtaining the test earlier than the chronological age when the preventative test is typically recommended. For example, if the user is chronologically 43 years old, but determined to be biologically 46 years old, then a recommendation may be made for the user to consult with a doctor about getting a colonoscopy earlier than typical which is typically recommended to be done at the age 45.

As indicated in FIG. 8, after some time, the process can return to block 802 and the user's age can be predicted again to determine biological age of the user and the user's age gap to obtain another age gap determination at a later time than the first time. This process can be repeated as desired to obtain multiple age gap determinations.

At block 816, an age gap rate of change can be determined. In the case where the user has obtained multiple time-separated age gap determinations, the age gaps can be compared to one another to determine an age gap rate of change. For example, a first age gap may be determined to be 2. If a second age gap determined a year later is 2.5, then the age gap rate of change is 0.5 per year. If the second age gap was determined to be 1.9, then the age gap rate of change is −0.1 per year. The age gap rate of change can be used as an approximation of whether a user is getting healthier or not. For example, if the age gap rate of change is 0 or a negative number, then the user may be getting healthier by practicing lifestyle changes, losing weight, healing from disease, and so forth. On the other hand, if the age gap rate of change is positive, then that may indicate that a user may be getting less healthy and/or that a recommended intervention was unsuccessful. The user may be asked about the user's compliance with a recommended intervention. Alternative recommendations or interventions than previously provided can be provided to the user.

It is understood that the age gap rate of change correlates to a rate of change of the biological age and a rate of change of the chronological age. For example, a biological rate of change can be calculated between a previously determined first predicted age and the second predicted age, and a comparison can be provided between the biological rate of change and a corresponding chronological rate of change. When the biological rate of change is greater than the chronological rate of change, this indicates a positive age gap rate of change, and a notification or recommendation can be provided as noted above. Similarly, subsequent biological rates of change can be determined over several determinations of a predicted biological age.

After determining an age gap rate of change, then returning to block 814, a notification of the age gap rate of change and/or recommendations may be provided. For example, the user may be provided recommendations similar to those described above. In the case that the age gap rate of change has not increased, then the user can be notified with an encouraging message. In the case that the age gap rate has increased, this could indicate that a recommendation or recommended intervention was unsuccessful or ineffective and an alternative recommendation or recommended intervention may be suggested. If the age gap rate has increased unexpectedly, a recommendation may be provided to the user to consult a physician.

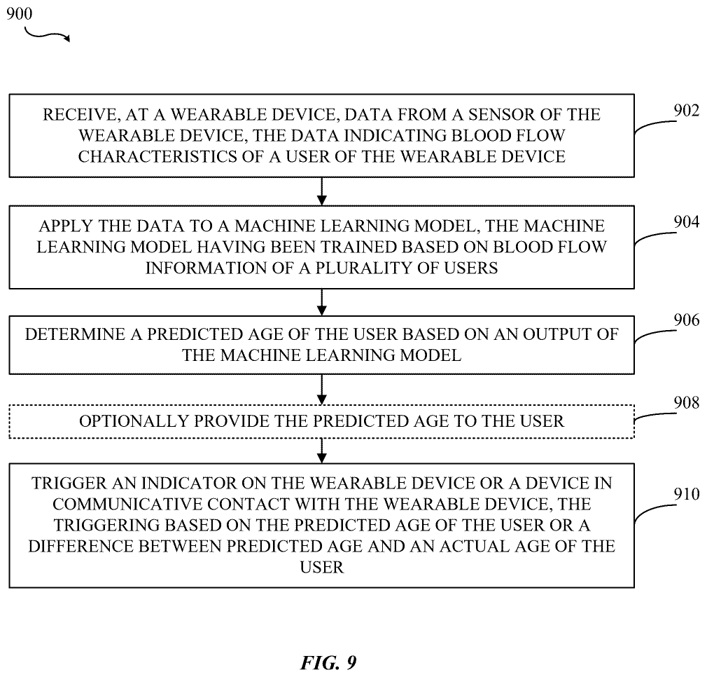

FIG. 9 illustrates a flow diagram of an example process 900 for utilizing a machine learning model to predict biological age of a user based on physiological signals, in accordance with some implementations. One or more blocks (or operations) of the process flow diagram of FIG. 9 may be performed by one or more other components and other suitable devices. Further for explanatory purposes, the blocks are described herein as occurring in serial, or linearly. However, multiple blocks of the process may occur in parallel. In addition, the blocks of the process need not be performed in the order shown and/or one or more blocks of the process need not be performed and/or can be replaced by other operations.

At block 902, the process 900 may include receiving, at a wearable device, data from a sensor of the wearable device, the data indicating blood flow characteristics of a user of the wearable device. As noted above, the data may be from a PPG sensor, for example, or other suitable physiological signal sensors.

At block 904, process 900 may include applying the data to a machine learning model, the machine learning model having been trained based on blood flow information of a plurality of users. The machine learning model may be trained, for example, using a subset of data from participants in a health study as noted above. The training data, however, may come from other sources instead.

At block 906, process 900 may include determining a predicted age of the user based on an output of the machine learning model. As noted above, the predicted age may be determined to correspond to a biological age of the user, or a biological age of the user may be based on the predicted age.

At block 908, process 900 may optionally include providing the predicted age to the user. For example, the user may be notified of the predicted age or may be provided a recommendation for health intervention based upon the predicted age. In some implementations, the user may not be provided their predicted age directly, but may be provided some indicator that the predicted age was close to their chronological age or that the predicted age was divergent from their chronological age, such as provided at block 910.

At block 910, process 900 may include triggering an indicator on the wearable device or a device in communicative contact with the wearable device, the triggering based on the predicted age of the user or a difference between predicted age and an actual age of the user. For example, if an age gap between the predicted age, e.g., the biological age, and the actual age, e.g., chronological age of the user, is above a threshold, then a trigger may occur to provide a health indicator on the wearable device or on another one of the user's devices associated with the wearable device or associated with the user's account. The health indicator may also include recommendations for interventions, such as described above.

As described above, one aspect of the present technology is the gathering and use of data available from specific and legitimate sources for predicting a physiological state of a user from physiological signals from wearable electronic devices using large-scale training of foundation models. The present disclosure contemplates that in some instances, this gathered data may include personal information data that uniquely identifies or can be used to identify a specific person. Such personal information data can include audio data, demographic data, location-based data, online identifiers, telephone numbers, email addresses, home addresses, biometric data or records relating to a user's health or level of fitness (e.g., vital signs measurements, medication information, exercise information, motion information, heartrate information workout information), date of birth, or any other personal information.

The present disclosure recognizes that the use of such personal information data, in the present technology, can be used to the benefit of users. For example, the personal information data can be used for capturing personal biometric data including physiological signals.

The present disclosure contemplates that those entities responsible for the collection, analysis, disclosure, transfer, storage, or other use of such personal information data will comply with well-established privacy policies and/or privacy practices. In particular, such entities would be expected to implement and consistently apply privacy practices that are generally recognized as meeting or exceeding industry or governmental requirements for maintaining the privacy of users. Such information regarding the use of personal data should be prominently and easily accessible by users and should be updated as the collection and/or use of data changes. Personal information from users should be collected for legitimate uses only. Further, such collection/sharing should occur only after receiving the consent of the users or other legitimate basis specified in applicable law. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations which may serve to impose a higher standard. For instance, in the US, collection of or access to certain health data may be governed by federal and/or state laws, such as the Health Insurance Portability and Accountability Act (HIPAA); whereas health data in other countries may be subject to other regulations and policies and should be handled accordingly.

Despite the foregoing, the present disclosure also contemplates aspects in which users selectively block the use of, or access to, personal information data. That is, the present disclosure contemplates that hardware and/or software elements can be provided to prevent or block access to such personal information data. For example, in the example of large-scale training of foundation models for predicting a physiological state of a user from physiological signals on wearable electronic devices, the present technology can be configured to allow users to select to “opt in” or “opt out” of participation in the collection and/or sharing of personal information data during registration for services or anytime thereafter. In addition to providing “opt in” and “opt out” options, the present disclosure contemplates providing notifications relating to the access or use of personal information. For instance, a user may be notified upon downloading an application that their personal information data will be accessed and then reminded again just before personal information data is accessed by the application.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing identifiers, controlling the amount or specificity of data stored (e.g., collecting location data at city level rather than at an address level or at a scale that is insufficient for facial recognition), controlling how data is stored (e.g., aggregating data across users), and/or other methods such as differential privacy.

Therefore, although the present disclosure broadly covers use of personal information data to implement one or more various disclosed implementations, the present disclosure also contemplates that the various implementations can also be implemented without the need for accessing such personal information data. That is, the various implementations of the present technology are not rendered inoperable due to the lack of all or a portion of such personal information data.

FIG. 10 illustrates an electronic system 1000 with which one or more implementations of the subject technology may be implemented. The electronic system 1000 can be, and/or can be a part of, the electronic device 110, and/or the server 120 shown in FIG. 1. The electronic system 1000 may include various types of computer readable media and interfaces for various other types of computer readable media. The electronic system 1000 includes a bus 1008, one or more processing unit(s) 1012, a system memory 1004 (and/or buffer), a ROM 1010, a permanent storage device 1002, an input device interface 1014, an output device interface 1006, and one or more network interfaces 1016, or subsets and variations thereof.

The bus 1008 collectively represents all system, peripheral, and chipset buses that communicatively connect the numerous internal devices of the electronic system 1000. In one or more implementations, the bus 1008 communicatively connects the one or more processing unit(s) 1012 with the ROM 1010, the system memory 1004, and the permanent storage device 1002. From these various memory units, the one or more processing unit(s) 1012 retrieves instructions to execute and data to process in order to execute the processes of the subject disclosure. The one or more processing unit(s) 1012 can be a single processor or a multi-core processor in different implementations.

The ROM 1010 stores static data and instructions that are needed by the one or more processing unit(s) 1012 and other modules of the electronic system 1000. The permanent storage device 1002, on the other hand, may be a read-and-write memory device. The permanent storage device 1002 may be a non-volatile memory unit that stores instructions and data even when the electronic system 1000 is off. In one or more implementations, a mass-storage device (such as a magnetic or optical disk and its corresponding disk drive) may be used as the permanent storage device 1002.

In one or more implementations, a removable storage device (such as a flash drive, and its corresponding solid-state drive) may be used as the permanent storage device 1002. Like the permanent storage device 1002, the system memory 1004 may be a read-and-write memory device. However, unlike the permanent storage device 1002, the system memory 1004 may be a volatile read-and-write memory, such as random-access memory. The system memory 1004 may store any of the instructions and data that one or more processing unit(s) 1012 may need at runtime. In one or more implementations, the processes of the subject disclosure are stored in the system memory 1004, the permanent storage device 1002, and/or the ROM 1010. From these various memory units, the one or more processing unit(s) 1012 retrieves instructions to execute and data to process in order to execute the processes of one or more implementations.

The bus 1008 also connects to the input device interface 1014 and output device interface 1006. The input device interface 1014 enables a user to communicate information and select commands to the electronic system 1000. Input devices that may be used with the input device interface 1014 may include, for example, alphanumeric keyboards and pointing devices (also called “cursor control devices”). The output device interface 1006 may enable, for example, the display of images generated by electronic system 1000. Output devices that may be used with the output device interface 1006 may include, for example, printers and display devices, such as a liquid crystal display (LCD), a light emitting diode (LED) display, an organic light emitting diode (OLED) display, a flexible display, a flat panel display, a solid state display, a projector, or any other device for outputting information. One or more implementations may include devices that function as both input and output devices, such as a touchscreen. In these implementations, feedback provided to the user can be any form of sensory feedback, such as visual feedback, auditory feedback, or tactile feedback; and input from the user can be received in any form, including acoustic, speech, or tactile input.

Finally, as shown in FIG. 10, the bus 1008 also couples the electronic system 1000 to one or more networks and/or to one or more network nodes, such as the electronic device 110 shown in FIG. 1, through the one or more network interface(s) 1016. In this manner, the electronic system 1000 can be a part of a network of computers (such as a LAN, a wide area network (“WAN”), or an Intranet, or a network of networks, such as the Internet. Any or all components of the electronic system 1000 can be used in conjunction with the subject disclosure.

Implementations within the scope of the present disclosure can be partially or entirely realized using a tangible computer-readable storage medium (or multiple tangible computer-readable storage media of one or more types) encoding one or more instructions. The tangible computer-readable storage medium also can be non-transitory in nature.

The computer-readable storage medium can be any storage medium that can be read, written, or otherwise accessed by a general purpose or special purpose computing device, including any processing electronics and/or processing circuitry capable of executing instructions. For example, without limitation, the computer-readable medium can include any volatile semiconductor memory, such as RAM, DRAM, SRAM, T-RAM, Z-RAM, and TTRAM. The computer-readable medium also can include any non-volatile semiconductor memory, such as ROM, PROM, EPROM, EEPROM, NVRAM, flash, nvSRAM, FeRAM, FeTRAM, MRAM, PRAM, CBRAM, SONOS, RRAM, NRAM, racetrack memory, FJG, and Millipede memory.

Further, the computer-readable storage medium can include any non-semiconductor memory, such as optical disk storage, magnetic disk storage, magnetic tape, other magnetic storage devices, or any other medium capable of storing one or more instructions. In one or more implementations, the tangible computer-readable storage medium can be directly coupled to a computing device, while in other implementations, the tangible computer-readable storage medium can be indirectly coupled to a computing device, e.g., via one or more wired connections, one or more wireless connections, or any combination thereof.

Instructions can be directly executable or can be used to develop executable instructions. For example, instructions can be realized as executable or non-executable machine code or as instructions in a high-level language that can be compiled to produce executable or non-executable machine code. Further, instructions also can be realized as or can include data. Computer-executable instructions also can be organized in any format, including routines, subroutines, programs, data structures, objects, modules, applications, applets, functions, etc. As recognized by those of skill in the art, details including, but not limited to, the number, structure, sequence, and organization of instructions can vary significantly without varying the underlying logic, function, processing, and output.

While the above discussion primarily refers to microprocessor or multi-core processors that execute software, one or more implementations are performed by one or more integrated circuits, such as ASICs or FPGAs. In one or more implementations, such integrated circuits execute instructions that are stored on the circuit itself.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. Skilled artisans may implement the described functionality in varying ways for each particular application. Various components and blocks may be arranged differently (e.g., arranged in a different order, or partitioned in a different way) all without departing from the scope of the subject technology.

It is understood that any specific order or hierarchy of blocks in the processes disclosed is an illustration of example approaches. Based upon design preferences, it is understood that the specific order or hierarchy of blocks in the processes may be rearranged, or that all illustrated blocks be performed. Any of the blocks may be performed simultaneously. In one or more implementations, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

As used in this specification and any claims of this application, the terms “base station”, “receiver”, “computer”, “server”, “processor”, and “memory” all refer to electronic or other technological devices. These terms exclude people or groups of people. For the purposes of the specification, the terms “display” or “displaying” means displaying on an electronic device.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The predicate words “configured to”, “operable to”, and “programmed to” do not imply any particular tangible or intangible modification of a subject, but, rather, are intended to be used interchangeably. In one or more implementations, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

Phrases such as an aspect, the aspect, another aspect, some aspects, one or more aspects, an implementation, the implementation, another implementation, some implementations, one or more implementations, an embodiment, the embodiment, another embodiment, some implementations, one or more implementations, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

The word “exemplary” is used herein to mean “serving as an example, instance, or illustration”. Any embodiment described herein as “exemplary” or as an “example” is not necessarily to be construed as preferred or advantageous over other implementations. Furthermore, to the extent that the term “include”, “have”, or the like is used in the description or the claims, such term is intended to be inclusive in a manner similar to the term “comprise” as “comprise” is interpreted when employed as a transitional word in a claim.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for”.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but are to be accorded the full scope consistent with the language claims, wherein reference to an element in the singular is not intended to mean “one and only one” unless specifically so stated, but rather “one or more”. Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the subject disclosure.

What is claimed is:

1. A method comprising:
receiving, at a wearable device, data from a sensor of the wearable device, the data indicating physiological characteristics of a user of the wearable device;
applying the data to a machine learning model, the machine learning model having been trained based on physiological information of one or more users;
determining a predicted age of the user based on an output of the machine learning model; and
triggering an indicator on the wearable device or a device in communicative contact with the wearable device, the triggering being based on the predicted age of the user or a difference between predicted age and a chronological age of the user.

2. The method of claim 1, further comprising:
comparing the predicted age of the user to a chronological age of the user to determine a health indicator; and
providing the health indicator to the user.

3. The method of claim 2, further comprising:
obtaining health or activity information from the user or the wearable device; and
correlating the health or activity information with the health indicator.

4. The method of claim 1, further comprising:
filtering the received data based on sensor data from a second sensor of the wearable device.

5. The method of claim 1, wherein the physiological characteristics include at least one of blood flow, heart rate, heart rhythm, heartbeat strength, or heartbeat timing.

6. The method of claim 1, wherein the sensor comprises a photoplethysmography sensor or an electrocardiogram sensor.

7. The method of claim 1, further comprising:
associating a difference in the predicted age and the chronological age of the user to a health condition or a user behavior.

8. The method of claim 7, wherein the health condition or the user behavior comprises smoking, diabetes, or a heart condition.

9. The method of claim 1, wherein the predicted age is a second predicted age, further comprising:
providing a comparison of the second predicted age to a previously determined first predicted age.

10. The method of claim 1, wherein the predicted age is a second predicted age, further comprising:
calculating a biological rate of change between a previously determined first predicted age and the second predicted age; and
providing a comparison between the biological rate of change and a corresponding chronological rate of change.

11. The method of claim 10, wherein when the biological rate of change is greater than the corresponding chronological rate of change, providing a notification to the user.

12. The method of claim 10, wherein the biological rate of change is a second biological rate of change, the method further comprising:
calculating a first biological rate of change between a previously determined third predicted age and the previously determined first predicted age;
comparing the first biological rate of change to the second biological rate of change;
based on the second biological rate of change being less than the first biological rate of change, providing a first notification to the user; and
based on the second biological rate of change being greater than the first biological rate of change, providing a second notification to the user.

13. The method of claim 10, further comprising:
determining an effectiveness of a previously provided intervention recommendation based on the difference between the biological rate of change and the corresponding chronological rate of change; and
based on the difference between the biological rate of change being greater than the corresponding chronological rate of change, providing a different intervention recommendation than the previously provided intervention recommendation.

14. The method of claim 1, wherein the data from the sensor of the wearable device is sampled over a plurality of time periods and combined.

15. The method of claim 1, further comprising:
training the machine learning model, comprising:
receiving candidate data having a first plurality of records;
forming a second plurality of records from the first plurality of records by including in the second plurality of records only those records corresponding to healthy individuals;
forming a third plurality of records from the second plurality of records by taking a random sampling from the second plurality of records; and
training the machine learning model on a subset of the candidate data corresponding to the third plurality of records.

16. The method of claim 15, wherein the subset of the candidate data includes sensor data from a first time period for each of the third plurality of records, wherein the subset of the candidate data includes questionnaire response data, wherein at least one of the first time periods occurs before a corresponding date of the questionnaire response data.

17. The method of claim 1, wherein the one or more users corresponds to a plurality of users and the plurality of users is a subset of a larger plurality of users, selected for training the machine learning model based on one or more common characteristics indicating that the plurality of users are healthy.

18. A device comprising:
a memory; and
one or more processors configured to:
receive data from a sensor, the data indicating physiological characteristics of a user of the device;
apply the data to a machine learning model, the machine learning model having been trained based on physiological information of one or more users;
determine a predicted age of the user based on an output of the machine learning model; and
trigger an indicator on the device or another device in communicative contact with the device, the triggering being based on the predicted age of the user or a difference between predicted age and a chronological age of the user.

19. The device of claim 18, wherein the one or more processors are further configured to:
compare the predicted age of the user to a chronological age of the user to determine a health indicator; and
provide the health indicator to the user.

20. A non-transitory computer-readable medium storing instructions thereon, which when executed cause one or more processors to perform a process including:
receiving, at a wearable device, data from a sensor of the wearable device, the data indicating physiological characteristics of a user of the wearable device;
applying the data to a machine learning model, the machine learning model having been trained based on physiological information of one or more users;
determining a predicted age of the user based on an output of the machine learning model; and
triggering an indicator on the wearable device or a device in communicative contact with the wearable device, the triggering being based on the predicted age of the user or a difference between predicted age and a chronological age of the user.
